(19) United States
(12) Patent Application Publication
(10) Pub. No.: US 2023/0014952 A1
(43) Pub. Date: Jan. 19, 2023

(54) RETINAL IMAGING SYSTEM

(71) Applicant: SPRING VISION LTD., Haifa (IL)

(72) Inventors: Avihu Meir GAMLIEL, Pardes-Hana (IL); Noam ALLON, Haifa (IL)

(21) Appl. No.: 17/791,052

(22) PCT Filed: Jan. 6, 2020
(86) PCT No.: PCT/IL2021/050021

§ 371 Date: Jul. 6, 2022

Related U.S. Application Data
(60) Provisional application No. 62/957,484, filed on Jan. 6, 2020.

(51) Int. Cl. A61B 3/12; A61B 3/14; A61B 3/00; A61B 90/00

(52) U.S. Cl. CPC A61B 3/12; A61B 3/14; A61B 3/0083; A61B 3/0091; A61B 3/0033; A61B 3/0008; A61B 3/0075; A61B 90/03; A61B 2560/0233; A61B 2090/065

(57) ABSTRACT

A retinal imaging system is provided. The system comprises: a fundus camera having a focusing mechanism; an imaging module configured for imaging user's face and eyes and providing image date indicative of a relative orientation between an optical axis of the fundus camera and a line of sight of user's eye at user's eye target position; a position and alignment system configured and operable to utilize the image data indicative of said relative orientation for positioning the fundus camera at an operative position such that the optical axis substantially coincides with the line of sight of user's eye, to enable focusing the fundus camera on the retina; a sensing system comprising one or more sensors, configured and operable for monitoring a user's face position with respect to a predetermined registration position and generating corresponding sensing data; and a safety controller configured and operable to be responsive to the sensing data, and upon identifying that the user's face position with respect to the predetermined registration position

### TECHNOLOGICAL FIELD

The present invention is in the field of medical applications, and relates to a retinal imaging system and method.

### BACKGROUND

Retinal imaging systems typically utilize a fundus camera, which images the rear of the eye through the pupil and typically uses illumination and imaging optics with a common optical path. During the imaging procedure, the fundus camera is operated by an operator (specialist), being a technician or physician, as the case may be. The operator has to align and focus the fundus camera properly on the patient's pupil. To this end, the patient's head is kept steady in the chinrest and headrest of the fundus camera; and the operator first assess the “field of the eye”, and then moves the camera from side to side to ascertain the width of the pupil and the focusing peculiarities of the particular cornea and lens. The operator inspects the eye through the camera lens, moving the camera back and forth and up and down, looking for fundus details (e.g., retinal blood vessels), and then determines the single best position from which to acquire images of the retina. A working distance, being a distance between the pupil and the fundus camera along the optical axis of the camera, should also be properly adjusted. That as a result of being too close to the eye, a bright, crescent-shaped light reflex appears at the edge of the viewing screen or a bright spot appears at its center, and if the camera is too far away, a hazy, poorly contrasted image results.

The procedure of camera location adjustment is time consuming, requiring involvement of a skilled operator, and also requiring the patient's patience while keeping his/her head steady in the chinrest and headrest of the fundus camera.

Various techniques have been developed for semi-automatic or automatic alignment/positioning of fundus camera and/or automatic focus adjustment of the fundus camera, and are described for example in the following patent publications: JP2010035728; US2008089480; CN110215186.

### GENERAL DESCRIPTION

There is a need in the art in a novel approach in professional retinal imaging enabling the use of a self-operated or at least semi-autonomous imaging system, that combines automatic alignment, positioning and focusing with safety control functions to perform efficient retinal imaging. Also, such a system should preferably be configured for self-calibration.

Such a self-operated and fully or partially autonomous retinal imaging system is particularly useful for eye checks of many people that are usually attentive. The system provides the results of eye inspection in an almost automatic fashion. These results (image data) can be further processed/analyzed using AI and Deep Learning methodology in order to reduce human (physician) involvement in the process. It should be understood that such an autonomous system is aimed at screening large amount of population, enabling automatic processing of image data to discern people with various retinal and systemic diseases.

As described above, a retinal imaging system typically includes a fundus camera mounted on a camera support assembly movable along at least vertical and two horizontal axes. As will be described further below, the system may utilize rotation of the fundus camera (or at least the optics therein) about one or more axes. Also, the fundus camera module typically includes a face cradle unit. In the conventional systems of the kind specified, a support plane, on which the fundus camera support and the face cradle unit are mounted, is a horizontal plane, and the face cradle unit includes a chinrest and headrest elements to keep the patient's head steady during the imaging session.

The inventors have found that such conventional configuration is less comfortable for a patient/user to properly place his/her face and keep it in a target position (fixation position) during retinal imaging, and moreover such configuration is practically unsuitable for autonomous or semi-autonomous system implementation. Thus, in some embodiments of the invention, the fundus camera assembly is configured such that an optical axis (central axis of field of view) of the fundus camera is tilted with respect to the horizontal plane, and a face support surface of a face cradle is appropriately tilted (e.g. is substantially perpendicular to the optical axis of the fundus camera) allowing the user to position his/her face such that it freely lays on the face support surface of the face cradle (avoiding any chinrest element) with the user's eyes pointing generally forward and downwards towards the field of view of the fundus camera.

The invention also preferably provides the use of a face contact frame projected from the face support surface, which allows making the face contact frame from a properly elastic and flexible material composition (e.g. rubber, silicone, etc.) making the entire procedure more comfortable for the user. The face contact frame (being elastic/flexible or not) may be removably mountable on/attachable to the face cradle, thus enabling it to be disposable or replaceable and easy to disinfect.

In some embodiments, the system of the invention provides for automatically adjusting position of a face cradle unit with respect to a fundus camera. Such adjustment might be needed to adjust the procedure to a specific user/patient. A typical example is that the users of different heights might require tunning of the face cradle position.

To this end, the system includes a face cradle unit position controller, and the face cradle unit is associated/equipped with a positioning mechanism (movement mechanism) controllably operable by data operational data provided by the controller to automatically adjust the position of the face cradle, e.g., based on estimated user data, such as user's height.

More specifically, the face cradle position controller is configured and operable to analyze image data of a scene including the region of interest acquired by an imaging module to detect the user's face in the image and estimate one or more user's parameters/conditions (e.g. the height) relative to standard average expected values of respective parameter/condition (e.g., height), and generate (if needed) position adjustment data to the movement mechanism of the face cradle unit. The latter utilizes this data to automatically adjust the face cradle position, i.e., its height with respect to the camera's field of view.

It should be understood, that for a self-operable retinal imaging system, in which various mechanical parts, or at least the fundus camera itself, is to be automatically moved with respect to the user's face while the face is at a registered position (e.g., freely lays on the face cradle and looking onto a so-called “fixation target”), it is important to provide a high-degree safety functionality of the system as well as a high-degree self-calibration functionality. Thus, according to the invention, the retinal imaging system includes an imaging module configured and operable to generate image data enabling registration of a line of sight (LOS) of user's eye at the user's eye target position, i.e. a fixation position or registration position enabling to bring the fundus camera to its alignment position with the user's line of sight, and includes a sensing system configured and operable to monitor a user's face position in the dedicated cradle (and possibly also with respect to a predetermined registration position with respect to the face contact frame) and also monitor a distance between the fundus camera and the user's face. The sensing system is associated with (connected to) a safety controller, which is responsive to the sensing data to monitor the degree of safety in the relative position between the user's face and the fundus camera.

The image data generated by the imaging module and the sensing data generated by the sensing system, as well as the sensing data analysis provided by the safety controller, are properly used to operate a positioning and alignment system of the fundus camera to bring and keep the fundus camera at an operative position such that its optical axis substantially coincides with the line of sight of user's eye and a working distance to the user's face is maintained. When the data analysis result is indicative of that alignment of the optical axis and/or working distance condition(s) appear(s) to be breached (either one of them does not satisfy the predetermined requirement), the system operates to stop the retinal imaging process and avoid any movement within the system.

With regard to self-calibration requirement, it should be noted that self-calibration is the process requiring reading of sensing data from the sensing system, where such sensing data relates to physical measures, such as distances, motor step size to linear dimension (e.g. millimeters) conversion, pixel to millimeters conversion, etc. Since in the self-operable system of the present invention there are moving parts, the self-calibration becomes more important in order to avoid increased positioning error after a while.

To achieve the user's eye target position, the imaging system includes a specifically designed fixation target (e.g. image, pattern) exposed to the user when his/her face is properly positioned on the face cradle. Practically, the system provides instructions to the user (audible and/or visual instructions). It should be understood that the autonomous or semi-autonomous system of the invention is suitable for use by people that are usually attentive.

The imaging module acquires images of the user's face, eyes, and irises, e.g., using IR illumination to detect the eye pupil, and generates corresponding image data indicative of a relative orientation of the line of sight of the user's eye (while at the user's eye target position) with respect to the optical axis of the fundus camera, and enabling to move the fundus camera to the aligned position at which its optical axis substantially coincides with the line of sight of user's eye.

Detection of the eye pupil is typically performed by video-based eye-trackers. A camera focuses on one or both eyes and records eye movement as the viewer looks at some kind of stimulus. Some of the known eye-trackers utilize detection of the center of the pupil and utilize infrared/near-infrared non-collimated light to create corneal reflections, such that a vector between the pupil center and the corneal reflections can be used to determine a reference point on a surface or the eye gaze direction. To this end, a simple calibration procedure of the user is usually needed before using the eye tracker. The suitable eye-tracking techniques based on infrared/near-infrared illumination may be the techniques known as bright-pupil and dark-pupil techniques, differing from one another in the location of an illumination source with respect to the light directing optics: with the illumination source being coaxial with the optical path, the eye acts as a retroreflector creating a bright pupil effect (similar to red eye); and with the illumination source being offset from the optical path, the pupil appears dark. Bright-pupil tracking creates greater iris/pupil contrast, allowing more robust eye-tracking with all iris pigmentation, and greatly reduces interference caused by eyelashes and other obscuring features; and also allows tracking in lighting conditions ranging from total darkness to very bright. The eye-tracking techniques are generally known, and although the system of the present invention may utilize any of the known suitable eye-tracking technique, this does not form part of the invention, and therefore needs not be described in more details.

Thus, according to one broad aspect of the present invention, there is provided a self-operable retinal imaging system comprising:

a fundus camera having a focusing mechanism;

an imaging module configured for imaging user's face and eyes and providing image date indicative of a relative orientation between an optical axis of the fundus camera and a line of sight of user's eye at user's eye target position;

a position and alignment system configured and operable to utilize the image data indicative of said relative orientation for positioning the fundus camera at an operative position such that the optical axis substantially coincides with the line of sight of user's eye, to enable focusing the fundus camera on the retina;

a sensing system comprising one or more sensors, configured and operable for monitoring a user's face position with respect to a predetermined registration position and generating corresponding sensing data; and a safety controller configured and operable to be responsive to the sensing data, and upon identifying that the user's face position with respect to the predetermined registration position corresponds to a predetermined risk condition, generating a control signal to the position and alignment system to halt movements of the fundus camera.

It should be noted that user's eye is to be brought to the fixation target position, corresponding to a predetermined orientation of the user eye's line of sight with respect to at least one predetermined target exposed to the user. In particular, such target position corresponds to intersection of the user eye's line of sight with a predetermined target (e.g. pattern) presented by the fundus camera.

The system may include a calibration mechanism configured and operable to perform self-calibration of the system. The self-calibration is aimed at detecting relative accommodation between an optical head of the fundus camera with respect to the user eye, and determining a distance (typically in a millimeter scale) that the optical head sis to be moved and a direction of such movement. To this end, calibration targets are used which are internal system targets such as two-dimensional element(s) and/or color pattern and/or QR codes, which are used for scene analysis in the vicinity of a region of interest.

Thus, the system utilizes fixation target(s) which are presented to the user in order to modify his line of sight orientation (move his eye in the requested position) that he will gaze in specific direction (in order to capture different part of his retina). The system may further use calibration target(s) of a different type for the scene analysis, i.e. determining whether and how the position of the optical head is to be tuned relative to the user's eye location.

It should be understood that such self-calibration might be needed, periodically or prior to each inspection stage, in order to avoid increased positioning error that might occur after a while. A calibration controller receives and analyzes the sensing data indicative of physical measures, such as distances, conversion of a motor step size to linear dimension (e.g. millimeters), pixel to millimeters conversion, etc., and identifies whether the target position has been changed from a nominal one in order to take this change into account for proper positioning of the fundus camera. Such self-calibration is more important in the self-operable system which utilizes moving parts.

For example, the system may utilize two targeting stages aimed at different purposes, which may be implemented using the common or different targets. The first targeting stage is aimed at the system self-calibration by image processing, and the second targeting stage is aimed at tracking the user's eye in order to enable capture different retinal areas/regions.

The self-calibration is performed by image processing and may for example be implemented using the target or physical elements serving as calibration element(s). Such elements may include one or more of the following: QR codes, color patterns, physical 2D or 3D shapes, etc. The calibration element(s) may be arranged within the system aside the face cradle or on the fundus camera, or on arear panel or anywhere within the system packaging.

Generally speaking, during the imaging session (by the fundus camera), the user may be asked/instructed to look at a small target presented by the fundus camera in order to capture different retinal areas. The natural eye movement that tracks the target enables the view line to the desired retinal area.

The retinal imaging system is associated with (i.e. includes or is connectable to) a control system which comprises inter alia a position controller configured and operable to be responsive to the image data and the sensing data to generate position and alignment data to said position and alignment system to perform controllable movements of the fundus camera to bring the fundus camera to the operative position; and a movement controller configured and operable to be responsive to the sensing data and to the control signal from the safety controller to operate the position and alignment system to halt the movements of the fundus camera.

The safety controller may be configured and operable to analyze the sensing data from one or more sensors of the sensing system indicative of a distance between the user's face and the fundus camera to enable generation of said control signal upon identifying a change in said distance corresponding to the risk condition. Preferably, such one or more sensors providing the distance data comprises at least one ultrasound sensor.

The position and alignment system comprises: a first driving mechanism operable in accordance with the alignment data for moving the fundus camera to a vertical aligned position of the optical axis corresponding to a vertical alignment with user's pupil; a second driving mechanism operable in accordance with the alignment data for moving the fundus camera to a lateral aligned position of the optical axis corresponding to substantial coincidence of the optical axis with the line of sight; and a third driving mechanism operable in accordance with the sensing data and a focal data of the fundus camera for moving the fundus camera along the optical axis to position a focal plane of the focusing mechanism at the retina of the user's eye. In some embodiments, the positioning system may be further configured for rotating the fundus camera in at least one plane.

The system comprises a registration assembly for registering a position of user's face with respect to the fundus camera. The registration assembly comprises a support platform defining a general support plane tilted with respect to a horizontal plane and carrying a face cradle defining a face support surface for supporting the user's face at the registered position during imaging such that user's eyes look general forward and downwards towards the fundus camera during retinal imaging. The face cradle preferably comprises a face contact frame projecting from said face support surface. The face contact frame may be made from elastic and flexible material composition. Alternatively or additionally, the face contact frame may be removable attachable to said face support surface to be disposable or replaceable.

The sensing system may comprise one or more sensors on the face cradle for monitoring a degree of contact of the user's face to the face support surface. Such one or more sensors on said face cradle may include at least one of the following: at least one pressure sensor, proximity sensor, or at least one IR sensor. Generally, one or more pressure sensors may be used to monitor the contact of the user's face with the face support surface. In some examples, at least three sensing elements may be used being located in three spaced-apart locations to monitor the degree of contact in respective at least three contact points with the face cradle.

The imaging module comprises one or more cameras (a pixel matrix detector) and is configured and operable to acquire images of a region of interest enabling to perform either naïve-approach image processing or direct 3D image acquisition. Thus, the imaging module may include at least two 2D imagers (cameras) with intersecting fields of view, or a 3D imager to generate the image data which is indicative of (allows determination of) the user's eye line of sight orientation with respect to the optical axis of the fundus camera. The camera(s) of the imaging module may be standalone unit(s) properly located with respect to the face cradle and the fundus camera and/or may be attached to/integral with the fundus camera.

In some embodiments, a single 2D camera can be used in combination with physical element(s) (e.g. target or calibration element), and this arrangement is calibrated to extract 3D positioning data of the optical system and the scene. Such physical elements can be QR codes, color patterns, physical 2D or 3D shapes, etc., that are positioned on the optical head and/or on various positions within the system packaging. In this implementation, there is no need to extract explicitly 3D data from image data, but 3D positioning data can be estimated using the size of the physical element(s), and perspective analysis (element's positioning and hiding).

The retinal imaging system preferably also comprises a user interface utility configured and operable to provide position and target instructions to the user. The position and target instructions correspond to registration of, respectively, the user's face position and orientation of the line of sight, and may comprise at least one of audio and visual instructions.

Preferably, an illumination system is provided being configured and operable to provide diffused (soft) light within a region of interest where the user's face is positioned during imaging by the fundus camera. Also preferably the diffused (soft) light has a temperature profile substantially not exceeding 4500° K. It should be noted that NIR illumination of about 780-940 nm can be used. It may be used for pupil detection. The illumination intensity/power is selected to be sufficient for the 2D imager operation.

In some embodiments, the system comprises a triggering utility configured and operable to be responsive to the alignment data and the distance data from the position controller and movement controller to generate a triggering signal to the fundus camera upon identifying that the alignment data and the distance data satisfy an operational condition.

The retinal imaging system may be associated with a control system, which is generally a computerized system including inter alia a data processor and analyzer, may be a part of the fundus camera or of a separate computer system configured and operable for data communication (e.g., wireless communication) with the imaging module, the sensing system, the positioning and alignment system and the fundus camera.

The control system may be further configured to apply AI and Deep Learning processing to the image data provided by the fundus camera to identify people with various retinal and systemic diseases, and generate data indicative of patient retinal condition and patient health condition. Alternatively, or additionally, the control system may be configured for data communication with a central station to transmit data indicative of the retinal image data obtained by the fundus camera to the central station for recording and further processing using AI and Deep learning methodology to determine patient retinal condition and patient health condition based on the image data obtained by the fundus camera. Generally, various functional utilities of the data processing software may be properly distributed between the control system associated with the fundus camera and a remote (central) data processing station. Such a central station may receive image data from multiple retinal imaging systems, configured according to the invention, and analyze such multiple measured data pieces to optimize the AI and Deep learning algorithms. Typically, the data processor may be associated with (has access to) a database storing various retinal image data pieces in association with corresponding retinal conditions and patient health conditions.

According to another broad aspect of the invention, there is provided a retinal imaging system comprising a face cradle and a fundus camera, wherein: the fundus camera is configured such that its optical axis is tilted with respect to a horizontal plane; and the face cradle defines a tilted face support surface for supporting a user's face in a free laying state with user's eyes looking forward and downwards towards a field of view of the fundus camera.

The fundus camera is associated with a position and alignment system configured as described above, enabling movement of the fundus camera with respect to said face cradle along at least three axes.

The face cradle preferably comprises a face contact frame projecting from said face support surface. The face contact frame may be made from elastic and flexible material composition. Alternatively, or additionally, the face contact frame may be removable attachable to said face support surface to be disposable or replaceable.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic block diagram of the constructional and functional parts of a retinal imaging system of the present invention;

FIG. 2 is a flow diagram of a method of operation of the retinal imaging system of the invention;

FIGS. 3 and 4A-4B is a schematic illustration of an exemplary embodiment of the configuration of the retinal imaging system of the invention; and

FIG. 5 exemplifies the configuration of the face cradle suitable for use in the system of the invention.

### DETAILED DESCRIPTION OF EMBODIMENTS

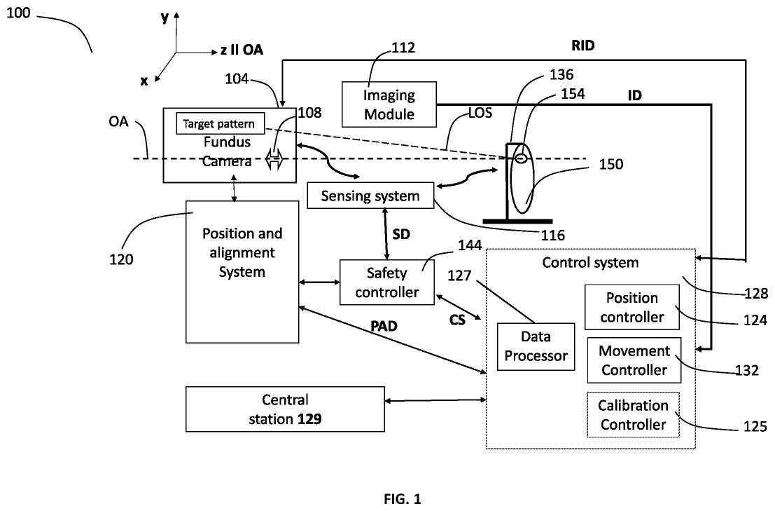

Referring to FIG. 1, there is a schematically illustrated a block diagram of the main constructional and functional parts of a retinal imaging system 100 of the present invention. The retinal imaging system 100 is configured as a self-operable system allowing a user to initiate and perform a retinal imaging session of his eye(s), while following instructions provided by the system. This enables to eliminate or at least significantly reduce any involvement of a technician or physician.

Data indicative of retinal images are properly stored and can be accessed by a physician for on-line or off-line analysis. For example, the stored data can be transmitted to a central computer station and be accessed from a remote device via a communication network using any known suitable communication techniques and protocols. As described above, the image data can be processed using AI and Deep Learning techniques.

The system 100 includes such main parts as a fundus camera 104, an imaging module 112, a sensing system 116, a position and alignment system 120, a safety controller 144, and a control system 128. The fundus camera 104 is typically positioned in association with a face cradle unit 136.

The configuration may be such that the face cradle is equipped with a movement mechanism which is controllably operable to move the cradle unit enabling automatic adjustment of its position to meet the requirements for a specific user/patient (e.g. take into account user's height difference from an average or nominal value).

Although not shown in this schematic illustration, the fundus camera and the face cradle may be mounted on a common support platform. As will be described further below, the invention also provides a novel configuration for the support platform.

As described above, the invention is aimed at providing a self-operable retinal imaging system which provides user-safety and effective retinal imaging. During the retinal imaging session, the user is requested/instructed to bring his face and eyes to a target position, by positioning his face on the face cradle and pointing his view to a target image presented by the fundus camera.

The imaging module 112 includes at least one imaging unit, which includes one or more imagers configured an operable to acquire images of user's face, eyes, irises and possibly also pupils (e.g., using appropriate eye tracking technique or eye and gaze tracking technique) and generate corresponding image data. As described above, the imaging module 112 may include one or more additional imaging units adapted for imaging the scene including a region of interest outside the fundus camera field of view and generating corresponding “external” image data, which can be used for self-calibration purposes. \Hence, the image data ID from the imaging module 112 may be also used for the self-calibration of the system, which may be implemented using the calibration target(s) in the form of QR codes, color patterns, physical 2D or 3D shapes, etc.). Further, while at the user's eye target position (as described above) the image data ID indicative of a relative orientation of an optical axis OA of the fundus camera with respect to the line of sight LOS of user's eye is analyzed. As described above the targets used at the self-calibration and imaging stages may or may not be the same.

Analysis of the image data ID is used to operate the position and alignment system 120 for positioning the fundus camera 104 at an operative position with a proper alignment of the optical axis OA of the fundus camera 104 such that it substantially coincides with the line of sight LOS of user's eye, while at said target position, and, while at the aligned position, to operate a focusing mechanism 108 of the fundus camera 104 to focus the fundus camera on the retina. To this end, the position and alignment system 120 is configured and operable for moving the fundus camera 104 along three axes with respect to the user's eye while at said user's eye target position.

The sensing system 116 is configured and operable for monitoring a relative position between a user's face 150 and the fundus camera 104 and generating corresponding sensing data SD. The sensing data is received and analyzed by a safety controller 144 to properly generate a control/alarm signal. Also, both the sensing data (or results of sensing data analysis) and the image data are used by the control system 128 to initiate (trigger) the retinal imaging session by the fundus camera and monitor the progression of the imaging session.

The control system 128 is a computer system including inter alia data input and output utilities, memory, and a data processor and analyzer. The data processor and analyzer comprises a position controller utility 124 (typically in software) configured and operable to be responsive to the image data ID from the imaging module 112 to generate position and alignment data PAD to the position and alignment system 120 to control the movements of the fundus camera to bring the fundus camera to the operative position. The position controller 124 also includes a calibration utility 125 configured and operable to utilize the image data to generate operational data to the position and alignment system to bring the fundus camera to the operational position.

As mentioned above, the face cradle may be associated with a movement mechanism enabling automatic adjustment of its position. To this end, the same position controller 124, or a separate controller of the control system 128, may be configured and operable to generate movement data to operate the movement mechanism of the face cradle to implement controllable movement of the face cradle to automatically adjust the position of the face cradle.

Such face cradle position controller may be responsive to image data ID from an imager, which may be that of the imaging module 120 or a separate imager (one or more 2D cameras), adapted to image a scene in the vicinity of a region of interest (i.e. vicinity of face cradle) to identify user's face in the image and generate corresponding estimated user's data, e.g. user's height relative to standard average expected height. Based on this estimate, the controller generates position adjustment data including movement data indicative of a movement required to be performed by the face cradle to automatically bring the face cradle to the proper position in association with a specific user, i.e., adjust the face cradle height with respect to the camera's field of view.

Also, the data processor and analyzer includes a movement controller 132 (typically in software) configured and operable to be responsive to the sensing data SD from the sensing system 116 to properly control the movement of the fundus camera to keep the required and safety working distance and responsive to signals from the safety controller 144. Hence, when the safety controller properly identifies that there exists/appears a predetermined risk condition in the relative position between the user's face and the fundus camera, it generate a corresponding control signal CS to the movement controller 132 which operates the position and alignment system to halt any movement of the fundus camera.

The safety controller 144 may be a separate processing unit or may be part of the control system 128. The safety controller is preprogrammed to determine whether position data, as well as movement data indicative of a predicted change in the position of the fundus camera relative to the user's face, arrived or is approaching a critical value corresponding to a risk condition, to properly generate the control signal CS. It should also be noted that the safety controller may utilize the sensing date to identify a change in the user's face position with respect to the face cradle and generate corresponding control/alarm signal, which may initiate generation of predetermined instructions to the user, together with and independent from the respective operation of the position and alignment system.

As also exemplified in the figure, the control system 128 includes a data processor 127 configured and operable to receive retinal image data RID from the fundus camera unit 104 and process this data to determine whether it is indicative of a specific anomality (disease). To this end, the data processor 127 is configured to apply AI and deep learning processing to the image data RID and utilize/access predetermined database storing various retinal image data pieces in association with corresponding retinal conditions (and corresponding individual's health condition). Alternatively, or additionally, the control system 128 may be configured for data communication with a central station 129 to transmit the raw data including retinal image data RID obtained by the fundus camera to the central station or transmit to the central station data indicative of the retinal image data resulting from some preprocessing performed by the data processor 127, for further processing at the central station using AI and deep learning techniques. The retinal image data RID and/or results of the processing of such data may be recorded at the control system 128 and/or at the central station 129. As described above, the central station 129 may be configured for communication with a plurality of retinal imaging systems, and analyze data received from these to optimize the AI and Deep learning algorithms as well as update the central database.

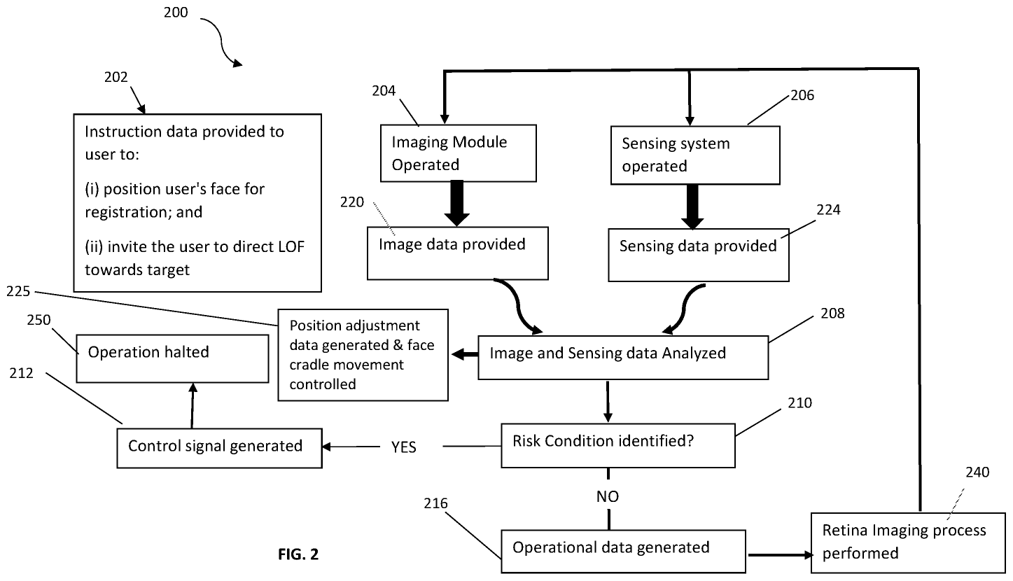

Referring to FIG. 2, there is schematically illustrated, by way of a flow diagram 200, a method of operation of the retinal imaging system of the invention. According to the method, instructions to the user are provided (step 202), preferably in the form of audio or visual format. More specifically, the user is instructed to position his/her face in a face cradle for registration and to look onto a target presented in the fundus camera. Such a target may be in the form of a visual mark, such as a picture or light pattern. Further, the imaging module and the sensing system are concurrently operated (steps 204 and 206) and provide, respectively, the image data (step) 220 and the sensing data (step 224) indicative of (enabling determination of), respectively, registration of user's face and eye position (including relative orientation of the line of sight) with respect to fundus camera, and degree of safety in the user's face position in the face cradle and that of the relative position of the fundus camera with respect to the user's face. The operation of the imaging module and the sensing system is initiated by the control unit, which may be performed in response to user's activation, e.g., by pressing a control button. Alternatively, or additionally, this can be automatically initiated by sensing element(s) of the sensing system, for example upon identifying that the user's face has been brought in contact with the face cradle.

In a next step, the image data and the sensing data, while being continuously provided, are continuously analyzed by a data processor and analyzing utility of the control system (step 208). The image data is initially indicative of the user's face position with respect to the face cradle and also with respect to the fundus camera (i.e., a relative orientation of the line of sight of user's eye, while pointing to the target) and the optical axis of the fundus camera (i.e., along the x- and y-axes), and possibly also is indicative of a distance between the user's face and the fundus camera). The sensing data is indicative of the proper contact between the user's face and the face cradle, and also of a distance between the user's face and the fundus camera. It should be understood that the distance determination may be performed in a double-check mode using both the image data of the imaging module and the sensing data of the sensing system.

The image data analysis may include generation of position adjustment data for the face cradle unit in association with a specific user/patient, in order to operate a movement mechanism of the face cradle unit to automatically adjust the position of the face cradle unit with respect to the fundus camera (step 225).

The image and sensing data analysis includes navigation/guidance data generation to the position and alignment system and a risk condition analysis/prediction to identify, while controlling position and movement steps, whether such navigation approaches a risk condition (step 210). With regards to the navigation procedure, it should be noted that position and alignment data analysis provides for bringing the fundus camera to the proper operational position, i.e., position of the alignment of the optical axis of the fundus camera with the user's eye line of sight and positioning of the so-aligned fundus camera at a required working distance to the user's eye. When the control system identifies such a proper operational position of the fundus camera, a triggering signal is generated which actuates an auto-focus and auto illumination managed by the fundus camera using any suitable auto-focusing technique, e.g., that typically used in imaging systems including fundus cameras. However, it should be noted that these processes of auto-focus and auto illumination are triggered (capturing triggered) by the control system upon identifying that the fundus camera, while being navigated, approaches the fundus camera working distance. From the point the system triggered the fundus camera, all its operations are fully automatic (focus, illumination, image processing, etc.).

If during navigation or later during the fundus camera operation (imaging session) a risk condition is identified, the control/alarm signal is generated (step 212) and movements (and possibly also operation) of the fundus camera are halted (step 250). Such a risk condition may be associated with exaggerated proximity of the fundus camera to the user's eye, and/or user's face movement from the registered position, and/or insertion of hands or other things in between the face cradle and the fundus camera. All such unsafety situations can be properly detected by the sensing system (e.g. ultrasound sensor(s)), which determines the distance between the fundus camera and the face cradle and detects obstacle at distance below the working distance. It should also be understood that the imaging module, i.e. the camera(s), can also detect any change towards a risk condition, thus performing together with the sensing system a double-check to keep the safety operation of the system.

As long as safety is maintained, i.e. risk condition is not identified, the process continues with generating operational data (step 216) and performing retinal imaging process (step 240). As the retinal imaging session proceeds, respective instructions are being provided to the user for directing the user's gaze towards the field of view of the fundus camera (e.g. towards the target) and maintaining user's face position and gaze (e.g. by instructing the user to keep the eyes open). The method is performing iteratively the steps above until the retinal imaging process is completed consecutively for the two eyes.

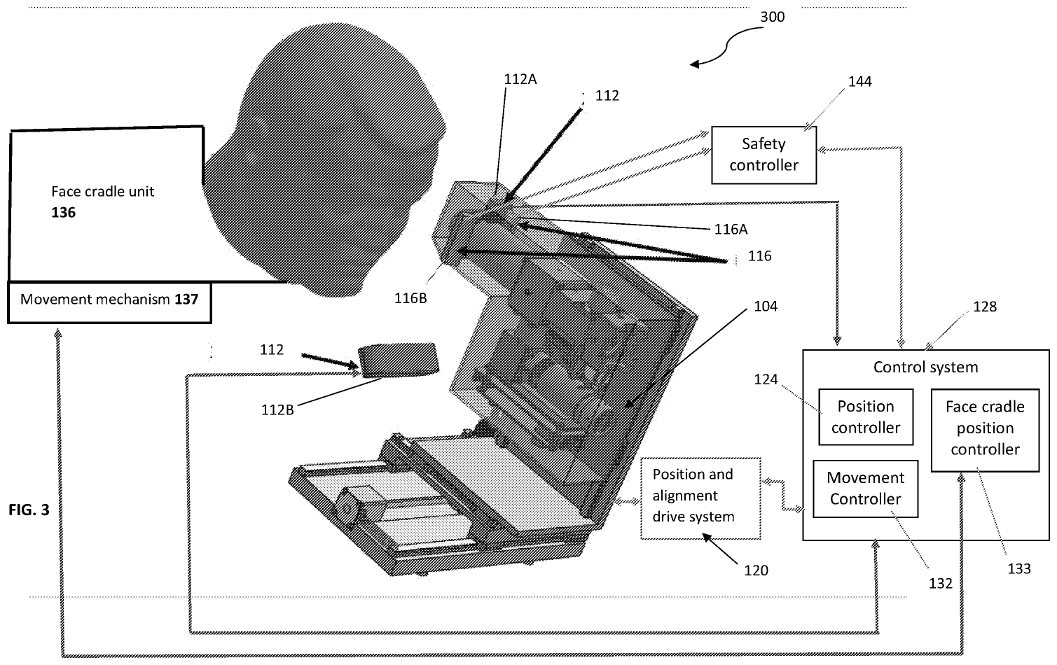

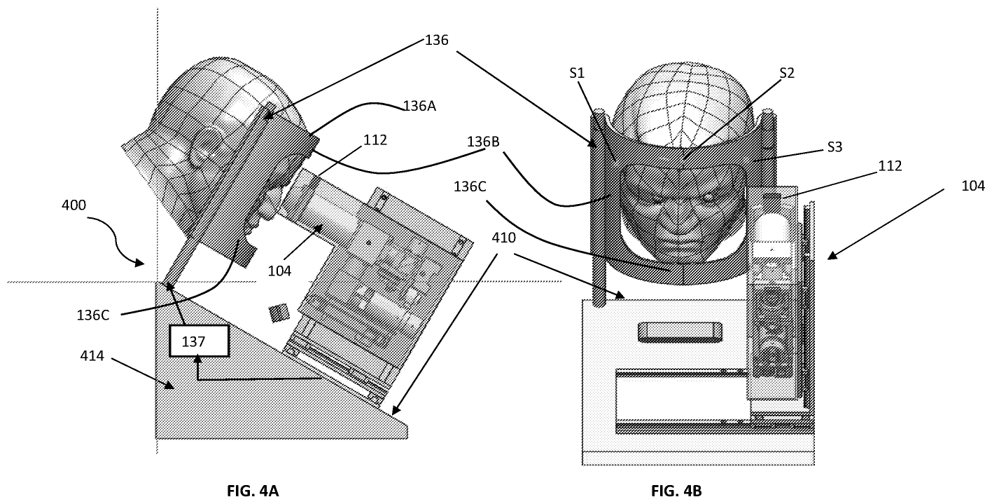

Reference is made to FIGS. 3 and 4A-4B illustrating a specific but not limiting example of the configuration and operation of the retinal imaging system 300 of the present invention. To facilitate understanding, the same reference numbers are used for identifying functionally similar elements of the exemplary system 300 and the above-described system 100 shown by the block diagram of FIG. 1.

As shown in FIG. 3, the retinal imaging system includes a fundus camera 104 associated with a face cradle unit (not shown here), which may be mounted on a common support platform with the fundus camera, as will be described further below. The system 300 further includes an imaging module 112 configured an operable as described above to acquire images of the user's face, eye, and iris/pupil and generate image data indicative of a relative orientation of a line of sight (LOS) of user's eye at the user's eye target position and the optical axis of the fundus camera 104. As shown in the figure, the imaging module 112 may include one or more imagers (cameras), which may be carried by the fundus camera module (as exemplified by imager(s) 112A) and/or separate (stand alone) imager(s) 112B. It should be understood that the imaging module preferably needs to provide 3D information about a region of interest being imaged. This can be achieved using any known suitable imagers' configurations. For example, two cameras with intersecting fields of view can be used; or a single camera with well-known physical targets with predefined measures can be used. For example, structured light illumination can be used in order to extract 3D parameters of the scene.

The image data can thus be used to identify whether the user's face is properly positioned and if not enable generation of instructions to the user; and identify whether the user is looking onto the target, and if not enable generation of instructions to the user. Also, the image data can be used by face cradle position controller 133 to determine whether and how the position of the face cradle 136 is to be adjusted, via movement mechanism 137, to bring the user's face to proper position with respect to the camera field of view and/or registration target.

Further, the image data is used to determine required movements of the fundus camera along x- and y-axes in the plane perpendicular to the optical axis of the fundus camera (and possibly also along the optical axis, or z-axis) to bring the fundus camera to the operative position with respect to the user's eye.

The system 300 further includes a sensing system 116 associated with a safety controller 144, configured and operable as described above with reference to FIG. 1. The configuration and operation of the sensing system 116 are aimed at providing (or, when used together with the image data, enhancing) the safety functionality to the system 300 operation. The sensing system 116 includes a distance detecting sensor(s), which preferably include(s) ultrasound sensor(s), optic sensor(s) and/or proximity sensor(s)—two distance detecting sensors 116A and 116B being shown in the present example.

As described above, and not specifically shown in FIG. 3, the sensing system preferably also includes one or more sensing elements for sensing the user's face contact with the face cradle. This may be achieved by using three sensing elements to control the contact at three spaced-apart points.

Further provided in the retinal imaging system 300 is a position and alignment system 120 including appropriate drive mechanisms performing displacement of the fundus camera with respect to the face cradle. Generally, the drive mechanisms provide movement of the fundus camera along three perpendicular axes, including two axes, x- and y-axes in the plane perpendicular to the optical axis of the camera and the z-axis being the optical axis of the fundus camera. It should be noted that an additional drive mechanism may be provided for rotation or pivotal movement of the fundus camera or at least its optical axis.

It should be noted that in the description the x- and y-axes are at times referred to as, respectively, horizontal and vertical axes. However, as mentioned above and will be described more specifically further below, the support plane supporting the fundus camera and the face cradle may be tilted with respect to the horizontal plane. In this case the x- and y-axes are respectively parallel and perpendicular to the support plane, and these terms should be interpreted and understood accordingly. Generally, the configuration may be such that the optical axis of the fundus camera, i.e. its field of view, is oriented at a certain angle (tilted) with respect to the horizontal plane “looking” in a generally forward and upward direction, and the face cradle is configured such that, when user's face is fixed on the face cradle user's field of view is oriented generally forward and downwards towards the field of view of the fundus camera.

The position and alignment system 120 operates by the operational data provided by the control system for bringing the fundus camera to an operative position (via navigation of its movements based on the analysis of the image and sensing data) such that the optical axis of the fundus camera substantially coincides with the line of sight of user's eye, while at said target position and the required working distance from the fundus camera, to keep the level of safety and enable focusing the fundus camera on the retina. As shown in the figure, the control system 128 is provided being in data communication with the imaging module 112, the safety controller 144 and possibly also directly with the sensing system 116, and data communication with the position and alignment system 120. The control system 128 is configured and operable as described above with reference to FIGS. 1 and 2.

It should be noted, although not specifically shown in the figure, that the retinal imaging system 300 may include or may be used with an illumination system configured and operable to provide diffused (soft) light and/or NIR illumination within a region of interest where the user's face is positioned during imaging by the fundus camera. The diffused (soft) light preferably has an appropriate temperature profile, e.g. substantially not exceeding 4500° K, and proper illumination intensity.

FIGS. 4A and 4B show more specifically an example of the configuration of a support platform 400 according to the invention. The support platform 400 is configured to define a general support plane 410 for a fundus camera 104 and a face cradle 136 such that the optical axis of the fundus camera is tilted with respect to a horizontal plane.

It should be understood that, generally, the fundus camera and the face cradle may or may not be mounted on the same physical surface, but the orientations of the user's gaze and the optical axis of the fundus are to be considered with respect to a predetermined general plane. Hence, the common support plane 410 may or may not be constituted by a physical surface. In this not limiting example this is achieved by placing the fundus camera 104 and the face cradle 136 on a tilted surface 410 (defining the general support plane) of a wedge element 414. This configuration allows the face cradle 136 to define a face support surface 136A properly inclined with respect to a vertical plane, such that user's face can be positioned on said surface 136A freely laying on the face support surface with the user's eyes pointing generally forward and downwards towards the optical axis of the fundus camera (while looing on the target).

As also schematically illustrated in the example of FIG. 4B, the face cradle 136 is preferably equipped with n (n>1) sensing elements of the sensing system. These may be contact sensing element(s) or proximity sensor(s) (e.g. utilizing piezo-elements or capacitive sensors). Preferably these are at least three sensing elements S1-S3 ensuring consistent sensing the position of the user's face on the face support surface.

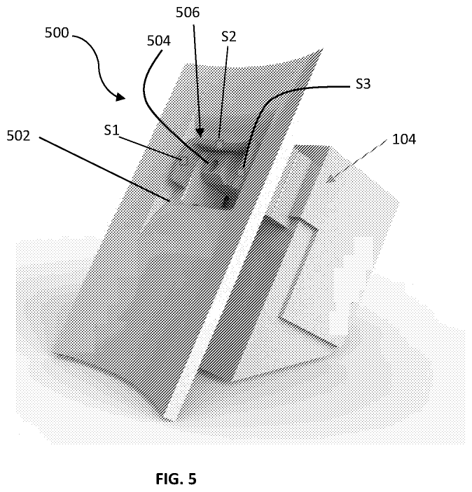

Although in this specific not limiting example of FIGS. 4A and 4B, the face cradle 136 includes a face frame 136B with a chinrest element 136C, the tilted configuration actually does not require and allows to avoid the need for the chinrest element. This is exemplified specifically in FIG. 5.

As shown schematically in FIG. 4A, the face cradle unit 136 may be associated with a movement mechanism 137, which is responsive to movement data generated at the control system associated with the camera unit (imaging module 112), as described above. Further, it should be understood that the face cradle and its movement mechanism may be configured to automatically adjust the face cradle position by implementing reciprocating movement of the face cradle unit with respect to the support surface, as well as varying an angular orientation of the face cradle.

FIG. 5 exemplifies the configuration of the face cradle 500 of the present invention advantageously suitable for use in the retinal imaging system, and specifically in the self-operable system of the kind specified. It should be noted, and also mentioned above, that the face cradle may or may not be mounted on the common support platform with the fundus camera. As shown in FIG. 5, the face cradle 500 has a face support surface 502, which is preferably (from the ergonomic/stability point of view) concave rather than planar, and is tilted with respect to a vertical plane to be properly positioned/mounted with respect to the fundus camera 104 whose optical axis is properly tilted from the horizontal plane. In the present example, the face cradle 500 and the fundus camera 104 form an integral unit.

The face support surface has an appropriate optical window 504 (e.g. opening) allowing imaging of user's eyes via the optical window. As also shown in the figure, the face cradle 500 may for example include a face contact frame 506 located on and projecting from the face support surface 502. The face contact frame 506 may be removably mountable on/attachable to the face cradle 500. Also, the face contact frame 506 may be made from a properly elastic and flexible material composition (e.g. rubber, silicone, etc.) making the entire procedure more comfortable for the user providing for ergonomic and more stable position during the imaging session. The face cradle may be equipped with one or more sensing elements—three such sensing elements S1, S2 and S3 being shown in this specific not limiting example. It should be understood, although not specifically shown, that the imaging module may be integral with/mounted on the fundus camera housing or may be a separate unit appropriately located to acquire the images of user's face, eye, iris, pupil. Also, the safety controller, as well as the control system may be integral with the fundus camera housing or may be stand alone device(s) connectable to the respective devices/units of the system as described above.

Thus, the present invention provides a novel configuration of the self-operable retinal imaging system, enabling a user to perform retinal imaging without a need for highly-skilled operator, and actually any operator's assistance, owing to the high-degree safety functionality of the system. The retina images may be stored in a memory of the control system, to be accessed by a skilled person for analysis; and/or may be communicated to an external control station. The invention also provides a novel face cradle configuration, as well as a novel configuration of an integral retina imaging system.

1. A retinal imaging system comprising:
a fundus camera having a focusing mechanism;
an imaging module configured for imaging user's face and eyes and providing image date indicative of a relative orientation between an optical axis of the fundus camera and a line of sight of user's eye at user's eye target position;
a position and alignment system configured and operable to utilize the image data indicative of said relative orientation for positioning the fundus camera at an operative position such that the optical axis substantially coincides with the line of sight of user's eye, to enable focusing the fundus camera on the retina;
a sensing system comprising one or more sensors, configured and operable for monitoring a user's face position with respect to a predetermined registration position and generating corresponding sensing data; and
a safety controller configured and operable to be responsive to the sensing data, and upon identifying that the user's face position with respect to the predetermined registration position corresponds to a predetermined risk condition, generating a control signal to the position and alignment system to halt movements of the fundus camera.

2. The system of claim 1, comprising a control system which comprises:
a position controller configured and operable to be responsive to the image data and the sensing data to generate position and alignment data to said position and alignment system to perform controllable movements of the fundus camera to bring the fundus camera to the operative position; and
a movement controller configured and operable to be responsive to the sensing data and to the control signal from the safety controller to operate the position and alignment system to halt the movements of the fundus camera.

3. The system of claim 1 or 2, wherein the safety controller is configured and operable to analyze the sensing data from one or more sensors of the sensing system indicative of a distance between the user's face and the fundus camera to enable generation of said control signal upon identifying a change in said distance corresponding to the risk condition.

4. The system of claim 3, said one or more sensors providing the distance data comprises at least one ultrasound sensor.

5. The system of claim 1, wherein said position and alignment system comprises:
a first driving mechanism operable in accordance with the alignment data for moving the fundus camera to a vertical aligned position of the optical axis corresponding to a vertical alignment with user's pupil;
a second driving mechanism operable in accordance with the alignment data for moving the fundus camera to a lateral aligned position of the optical axis corresponding to substantial coincidence of the optical axis with the line of sight; and
a third driving mechanism operable in accordance with the sensing data and a focal data of the fundus camera for moving the fundus camera along the optical axis to position a focal plane of the focusing mechanism at the retina of the user's eye.

6. The system of claim 5, wherein the position and alignment system further comprises a rotation mechanism for rotating the fundus camera with respect to at least one axis.

7. The system of claim 1, comprising a registration assembly for registering a position of user's face, said registration assembly comprising a face cradle for fixation of user's face at the registered position during imaging.

8. The system of claim 7, wherein said sensing system comprises one or more sensors on said face cradle for monitoring a degree of contact of the user's face to the face cradle.

9. The system of claim 8, wherein said one or more sensors on said face cradle include at least one of the following: at least one pressure sensor, or at least one IR sensor.

10. The system of claim 9, wherein said one or more sensors on said face cradle include at least one pressure sensor comprises at least three sensing elements located in three spaced-apart locations to monitor degree of contact in respective at least three contact points with the face cradle.

11. The system of claim 1, wherein said target position corresponds to a predetermined orientation of the user eye's line of sight with respect to at least one predetermined fixation target exposed to the user.

12. The system of claim 11, wherein said target position corresponds to intersection of the user eye's line of sight with the predetermined target presented by the fundus camera.

13. The system according to claim 1, further comprising at least one of the following:
a calibration mechanism configured and operable to perform self-calibration of the system, said calibration mechanism comprising at least one imager, one or more calibration targets located in a field of view of said at least one imager, and a calibration controller configured and operable to receive and analyze image data from said at least one imager and determine a relative position of an optical head of the fundus camera with respect a region of interest; and
an illumination system configured and operable to provide illumination within a region of interest where the user's face is positioned during imaging by the fundus camera.

14. The system according to claim 13, comprising said calibration mechanism, wherein said at least one calibration target includes at least one of the following: a two-dimensional element, color pattern, and QR codes.

15. The system of any one of the preceding claim 1, wherein the imaging module is characterized by at least one of the following: the imaging module comprises at least one imager; and the imaging module is configured and operable to image the user's eye using IR illumination to detect the eye pupil.

16. The system of claim 15, wherein the imaging module comprises the at least one imager and is characterized by at least one of the following: a) said at least one imager is configured as a 3D imager; and (b) the imaging module comprises two imagers with intersecting fields of view.

17. (canceled)

18. The system of claim 1, comprising a user interface utility configured and operable to provide position and fixation target instructions to the user.

19. The system of claim 18, characterized by at least one of the following: (i) said position and fixation target instructions correspond to registration of, respectively, the user's face position and orientation of the eye's line of sight and (ii) said position and fixation target instructions comprise at least one of audio and visual instructions.

20. (canceled)

21. The system according to claim 7, characterized by at least one of the following:
the imaging module is further configured and operable to provide image data indicative of one or more parameters of the user, the system further comprising a face cradle position controller configured and operable to the image data indicative of one or more parameters of the user and generate operational data to a movement mechanism of the face cradle to automatically adjust position of the face cradle with respect to the fundus camera based on said one or more parameters of the user;
the registration assembly is configured and operable for registering the position of user's face with respect to the fundus camera, the registration assembly comprising a support platform carrying the face cradle defining a face support surface for supporting the user's face at the registered position during imaging, the face support surface being tilted with respect to a vertical plane such that user's eyes look general forward and downwards towards the fundus camera.

22. The system according to claim 7, wherein the registration assembly is configured and operable for registering the position of user's face with respect to the fundus camera, the registration assembly comprising a support platform carrying the face cradle defining a face support surface for supporting the user's face at the registered position during imaging, the face support surface being tilted with respect to a vertical plane such that user's eyes look general forward and downwards towards the fundus camera, the system being further characterized by at least one of the following: (1) the fundus camera and the face cradle are mounted on the support platform; and (2) the face cradle comprises a face contact frame projecting from said face support surface.

23. (canceled)

24. (canceled)

25. The system of claim 22, wherein the face contact frame is characterized by at least one of the following: the face contact frame is made from elastic and flexible material composition; and the face contact frame is removably attachable to the face cradle, allowing the face contact frame to disposable or replaceable.

26. (canceled)

27. The system of claim 13, comprising said illumination system configured and operable to provide illumination within a region of interest where the user's face is positioned during imaging by the fundus camera, said illumination system being configured and operable to carry out one of the following: produce diffused (soft) light; and produce IR illumination.

28. (canceled)

29. (canceled)

30. The system according to claim 1, comprising at least one of the following:
a triggering utility configured and operable to be responsive to the position and alignment data and the distance data to generate a triggering signal to the fundus camera upon identifying that the position and alignment data and the distance data satisfy an operational condition; and
a data processor configured and operable to be responsive to retina image data from the fundus camera, and generate data indicative of a retinal condition and patient health condition.

31. (canceled)

32. (canceled)

33. The system according to claim 30, comprising the data processor configured and operable to be responsive to retina image data from the fundus camera, and generate data indicative of the retinal condition and patient health condition, the system being characterized by at least one of the following: said data processor is configured and operable to apply AI and deep learning processing to the retina image data; and the system is configured and operable to communicate with a remote station to transmit to the remote station data indicative of the retina image data.

34. (canceled)

35. A retinal imaging system comprising a face cradle and a fundus camera, wherein: the fundus camera is configured such that its optical axis is tilted with respect to a horizontal plane; and the face cradle defines a tilted face support surface for supporting a user's face in a free laying state with user's eyes looking forward and downwards towards a field of view of the fundus camera.

36. The support platform of claim 35, characterized by at least one of the following: the face cradle comprises a face contact frame projecting from said face support surface; the face contact frame is removably attachable to the face cradle, allowing the face contact frame to disposable or replaceable.

37. The support platform of claim 36, wherein the face cradle comprises a face contact frame projecting from said face support surface, the face contact frame being made from elastic and flexible material composition.

38. (canceled)
